(19) United States
(12) Patent Application Publication
(10) Pub. No.: US 2026/0210958 A1
(43) Pub. Date: Jul. 23, 2026

(54) DEVICE FOR HOLISTIC CHARACTERISATION OF THE SKIN-MICROBIOTA COMPLEX AND COSMETIC RECOMMENDATION METHOD

(71) Applicant: Byome Labs, Le Coudray (FR)

(72) Inventors: Thierry Bernardi, Pérignat-Les Sarliéve (FR); David Suissa, Fontenay-Sous-Bois (FR)

(21) Appl. No.: 19/140,231

(22) PCT Filed: Dec. 19, 2023
(86) PCT No.: PCT/EP2023/086672

§ 371 Date: Jun. 17, 2025

(30) Foreign Application Priority Data
Dec. 19, 2022 (FR) FR2213860

(51) Int. Cl. G01N 33/543; G01N 33/569; G06Q 30/0601

(52) U.S. Cl. CPC G01N 33/54388; G01N 33/56911; G01N 33/56938; G06Q 30/0631; G01N 2333/195; G01N 2333/31; G01N 2333/34; G01N 2469/10

(57) ABSTRACT

A field of characterizing the microbiota present on human skin. More particularly, it relates to a device for immunochromatographically revealing the skin-microbiota complex, which allows quick and meaningful biological determination of the two members of the skin-microbiota symbiosis by virtue of immunological characterization of biological markers accessible on the surface of the microbiota-skin interactome. Artificial intelligence is then used to recommend the best care suited, on the one hand, to the skin type and compatible, on the other hand, with the microbiota thereof, thereby reducing the risk of adverse effects. Moreover, in the event of skin imperfections and/or microbiota imbalance, the present disclosure recommends the best corrective treatment that is either guaranteed to be compatible with the microbiota or capable of regulating same.

### CROSS-REFERENCE TO RELATED APPLICATIONS

This application is a national phase entry under 35 U.S.C. § 371 of International Patent Application PCT/EP 2023/086672, filed Dec. 19, 2023, designating the United States of America and published as International Patent Publication WO 2024/133284 A1 on Jun. 27, 2024, which claims the benefit under Article 8 of the Patent Cooperation Treaty of French Patent Application Serial No. FR 2213860, filed Dec. 19, 2022.

### TECHNICAL FIELD

The present disclosure relates to the field of characterizing the microbiota present on human skin (or human skin “microflora”). More particularly, it relates to a device for immunochromatographically revealing the skin-microbiota complex, which allows quick and meaningful biological determination of the two members of the skin-microbiota symbiosis by virtue of immunological characterization of biological markers accessible on the surface of the microbiota-skin interactome. Artificial intelligence is then used to recommend the best care suited, on the one hand, to the skin type and compatible, on the other hand, with the microbiota thereof, thereby reducing the risk of adverse effects. Similarly, in the event of skin imperfections and/or microbiota imbalance, the present disclosure recommends the best corrective treatment that is either guaranteed to be compatible with the microbiota or capable of regulating same.

### BACKGROUND

Various solutions for analyzing skin microbiota are known in the state of the art.

WO 2014184151 A1 discloses a “point-of-care” diagnostic device based on lateral flow analysis technology and enables non-invasive analysis of factors secreted and diffusible from the skin surface. This document discloses, in particular, a diagnostic kit for detecting the presence or the quantity of one or more analytes in a test sample taken from a skin surface of a mammal, characterized in that the diagnostic kit comprises:
a) a separate insert for a lateral flow device comprising a membrane, the membrane having a defined thickness, width and length, optionally attached to a rigid support, and the separate insert being configured to obtain the test sample (analytes),
b) a lateral flow assay device configured to accept the separate insert, and
c) an attachment element configured to releasably attach the separate insert to the skin surface of a mammal.

Patent application US2022/0178943 provides a kit for detecting the presence or the quantity of one or more test analytes in a test sample obtained from a skin surface of a mammal, the kit comprising:
a) a lateral flow analysis (LFIA) device comprising a cassette comprising one or more porous elements forming an array of porous supports, wherein the cassette is configured to receive and hold a sample collection pad, wherein the sample collection pad is configured to make contact with the array of porous supports when the sample pad is inserted into the cassette,
b) a blister pack, wherein the blister pack contains a buffer solution, wherein the cassette is configured to receive the blister pack, and
c) a sample collection pad configured to be used to collect the test sample.

Application EP3691788A1 also relates to a diagnostic kit for detecting the presence or the quantity of one or more test analytes in a test sample obtained from a skin surface of a mammal, the diagnostic kit comprising:
a) a separate pad configured to be used to collect the test sample, the pad comprising a sample collection pad attached to a support member,
b) a lateral flow analysis device comprising one or more porous elements, wherein the lateral flow analysis device is configured to accept and hold the separate swab, wherein the sample collection pad is configured to form part of an array of porous supports when the separate swab is inserted into the lateral flow analysis device.

Application WO2019025610 discloses diagnostic kits and methods based on lateral flow assay devices to detect the presence or the quantity of one or more test analytes in a test sample taken from the skin of a mammal.

There are either analysis solutions based on highly precise molecular biology techniques, which are long and complicated to implement, and expensive, or lateral flow analysis solutions, which have only one or 2 markers, and which are not suited to skin characterization. Binary LFIA solutions only reveal the presence or the absence of biomarkers.

Since the methods used to recover the quantities of analytes sampled with the background art solutions (cotton buds or swabs, D-Squame or Sebutape adhesive strips, etc.) and the performance of lateral flow immunochromatographic tests require a minimum of the order of 10⁴ analytes per milliliter, genomic analysis solutions with a DNA amplification step are used to obtain significant results.

These solutions are not entirely satisfactory as they require complex post-sampling procedures (DNA extraction, PCR, Q-PCR or sequencing, etc.), in a biology laboratory and with personnel experienced in the use of genomics equipment.

To do this, they require extensive logistics with processing times between sampling and genomic analysis ranging from several days to several weeks, and are also relatively costly.

As a result, the number of studies evaluating the impact on the skin and its microbiota of cosmetic or personal care products applied to all or part of the face (or the rest of the body) remains very limited to date (for example, according to the clinicaltrial.gov website, in 2017-18, the study “The Effect of Skin Cleansers on the Skin Surface Microbiome,” whose sponsor was Mundipharma Manufacturing Pte Ltd, included 12 volunteers only, with analysis of their skin microbiome by Next Generation Sequencing, to compare the effects of 3 products: 1) 7.5% Povidone-Iodine, 2) 4% Chlorhexidine skin cleanser, and 3) Plain non-antibacterial soap.

Ultimately, the proposed metagenomic tests or analyses of the “microbiome” lead to the generation of a list of microorganisms identified for documentary purposes on an associated smartphone application. The ultimate objective of the approach consists of recommending cosmetics and personal care products based on the information collected via the smartphone application when the “skin microbiome” test is ordered (facial photos to assess parameters such as wrinkles, redness, etc., as well as declarative information such as age, weight, height, smoking habits, alcohol consumption, estimated skin condition—irritated, dry, oily—, declaration of pathologies—acne, eczema, atopic dermatitis, psoriasis etc.,—etc.).

### BRIEF SUMMARY

The field of the present disclosure is that of cosmetic care advice. Without inferring in the field of Health, a cosmetic product must be able to fulfill its role as a care product to maintain or improve the appearance of the user. When brought into contact with the various superficial parts of the human body (epidermis, hair and capillary systems, nails, lips and external genitalia), it must not present any health hazard. With the aim of providing personalized advice, the present disclosure proposes to provide information about the skin and the associated microbiota, corresponding to a symbiosis, by considering that a product must be suited to the needs of the skin in terms of hydration, hygiene, protection, modification of appearance (complexion, color, etc.) as well as to the susceptibility of the microbiota. For example, by recommending products containing active ingredients compatible with maintaining or regenerating the microbiota, and by avoiding active ingredients that destabilize the microbiota (favoring a microbial species that may induce a risk of opportunistic proliferation). By extension, the field of the present disclosure can be applied to the Medical Diagnosis of the skin of a patient, making it possible to consolidate the clinical diagnosis relating, for example, to the state of inflammation, allergic reaction (result of an immune system reaction) and to the microbiological status (balanced microbiota or dysbiosis with strong presence of an opportunistic pathogen). The present disclosure can then become an in vitro Diagnostic Medical Device. Similarly, by extension, the present disclosure can be used to monitor the condition of the skin over a given period and monitor the effect of a cosmetic routine, but also of a preventive and/or curative therapeutic treatment.

The field of the present disclosure applies to a human as a “mammal-microbe hybrid,” a “super-organism,” as the microbiota exceeds the number of cells making up the body by a factor of 10. The microbiota is thus understood to include all microorganisms in contact with the cells that make up the human body: viruses, phages, bacteria, yeasts, fungi, etc., right down to dust mites. If it is narrowed down to the bacterial microbiota, the order of magnitude comes down to a factor of 1.3. This situation of human-microbe symbiosis is common to the entire animal and plant world: a complex eukaryote-prokaryote relationship, refined, diversified and made more complex by reciprocal selective pressures, had to be established throughout evolution. The microbiota has multiple roles: it acts as a barrier against colonization by pathogens, has a maturing effect on the immune system, but also on the skin itself, and makes a major metabolic and nutritional contribution by providing an ability that does not exist in mammals for the hydrolysis of complex plant sugars, and the production of nutrients such as short-chain fatty acids and certain vitamins.

The field of the present disclosure considers two symbiotic partners together that have been considered separately for too long: the “skin” organ and the microbiota, long referred to as “skin flora.”

### The microbiota

The “skin flora” constitutes the external part of the microbiota of the human body (along with the microbiota of the digestive tract—the most important in terms of biomass—and of the oral, nasal, auricular and, in women, vaginal cavities). This flora acts as a barrier against external physical, chemical and biological aggressions, and interacts with the host organism depending on the site, generally via the cells of the immune system. Typically, bacterial biodiversity limits the risk of skin colonization by pathogenic bacteria and protects against inflammation, an allergic skin reaction.

Because of the microscopic size of the organisms that make it up, human microflora is invisible to the human eye. Adult skin harbors an average of 1 million bacteria per cm2 of skin, of over 500 different species. The skin naturally supports and maintains its own “micro-ecosystem.” This is formed at birth, then evolves until death. The skin microbiota is physically formed on the surface, and is a consortium of microbes organized in a biofilm. It feeds both on molecules and compounds excreted by the skin itself, and on compounds secreted by communities of more or less symbiotic microorganisms, mainly bacteria organized in a biofilm. The composition of the skin microbiota varies according to individuals, their age (newborn, adolescence, adulthood, old age), their gender (puberty, menopause in women), their activities, their behavior and the environment. The skin biofilm constantly renews itself, adapting to the natural desquamation and differentiated erosion of the skin, as well as to personal hygiene practices (which must not be excessive to protect the “barrier” functions of the skin, nor reduce the diversity of the microbiota that protects against inflammation). Furthermore, it evolves with age. In children, the bacterial biofilm differs according to the mode of delivery: by genital route, the child will be colonized by a community similar to the vaginal microbiota of the mother, whereas by Cesarean section it will be by a community similar to the skin microbiota. After 1 month, skin community profiles change steadily, gradually establishing the taxonomic specificities (viruses, bacteria, yeasts, fungi, and even mites) of the various differentiated sites on the human body: external sites on the skin (face, scalp, etc.), cavities (oral, nasal, vaginal), and internal sites (mainly the digestive tract). A recent study (Huang, 2020) even determined which body region (digestive tract, oral cavity, skin) of the microbiome could most accurately predict age and found that skin was the best, giving accurate predictions within 4 years on average. The importance of the level of symbiosis between the microbiota and the skin according to age is increasingly documented (Kim, 2019; Trojahn, 2015), confirming the need to ensure on the one hand that cosmetic products applied to the skin are compatible with the resident microbiota, and on the other hand that it may be wise to monitor the evolution of the human-microbiota symbiosis throughout a lifetime.

The microbiota plays a role in human skin odor, which also evolves throughout life. Odor production may derive from the degradation of sweat compounds such as volatile fatty acids or odoriferous steroids (James et al., FEMS Microbiology Ecology, 2013), with the involvement of certain bacterial genera such as Corynebacterium and strains affiliated to Streptococcus. Short-chain fatty acids, such as caprylic, capric, valerianic or propionic acid, are also involved in the development of body odor. These are degradation products of amino acids and long-chain fatty acids derived from sebum by bacteria of the genus Brevibacterium and Propionibacterium; propionic acid gives skin a hazelnut odor in low concentrations (typically after a shower) or the cheesy smell of dirty skin in high concentrations. These body odors vary based on gender, age or menstrual cycle.

The skin biofilm plays both a positive and a negative role. The formation of a commensal microbiota on the skin corresponds to colonization of groups of microorganisms having a protective role against other pathogenic microorganisms, which corresponds to an infection, can be identified by next-generation sequencing analysis techniques. For example, in the case of wound infections (extreme cases where the skin presents a discontinuity, a breach, instead of a homogeneous and continuous surface), groups of bacteria known as “pathogroups” (Proteus, Morganella, Anaerococcus and Peptoniphilus) have been described, the presence of which is correlated with deteriorating, non-healing wounds (Dunyach-Rémi et al. 2020). On the other hand, still in the case of wound infections, bacteria have been identified as exerting antimicrobial activity against pathogens (Nakatsuji et al., 2017), or reducing the virulence of pathogens (Ngba Essebe et al., 2017), thus protecting the host and giving rise to the notion of “positive flora.”

The microbiota resident on the skin is invisible except in cases where it provokes skin reactions from the immune system, for example, acne pimples linked to the proliferation of the Cutibacterium acnes bacterium, redness linked to an inflammatory state of eczema or atopic dermatitis exacerbated by the colonization of the bacterium Staphylococcus aureus, etc. There is also a correlation between spots on the skin and the presence of certain microorganisms, for example, of the genera Kocuria and Aerococcus (Zanchetta et al., 2022).

Microscopy has long been the main method for observing skin microbiota. Advances in biomolecular techniques have opened up new fields of study via genomics, molecular genetics, metagenomics, high-throughput sequencing and “culturomics.” Through direct sequencing of the DNA present in the sample, this approach not only provides a genomic description of the content of the sample, but also an insight into the functional potential of an environment. Biologists use high-throughput sequencing technologies to study metagenomic samples. The DNA sequences obtained are then analyzed using bioinformatics techniques. Metagenomic studies require logistics for transporting the sample taken from the person, usually with a simple cotton swab, to the analysis laboratory, which itself must be equipped with a range of equipment for extracting the genomic material (DNA) and sequencing same.

To reveal the presence of these microorganisms in biofilms, which are characteristic of a skin condition, it is also possible to use biological markers, molecules present on the surface of membranes, skin cell adhesion molecules and secreted molecules (exopolysaccharides, DNA, proteins, etc.) to form the matrix of the biofilm. These biological markers can be taken from the person and immediately analyzed using lateral flow immuno-assay (LFIA) techniques.

Thus, pathogenic microorganisms can be distinguished from commensals on the basis of pathogen-associated molecular patterns (PAMPs). These so-called “virulence” markers are the most extensively documented and described in the literature. These are secreted molecules, for example, the Panton-Valentine Leukocidin protein of Staphylococcus aureus, but also, with a lesser degree of virulence, porphyrins produced by Cutibacterium acnes, fluorescent molecules visible under ultra-violet light (Wood's lamp), which induce aggregation of Staphylococcus aureus and the expression of pro-inflammatory molecules by keratinocytes (IL-6, IL-8, prostaglandin E2, TNF-alpha, etc.).

Other markers are characteristic of the natural biofilm formation of microorganisms on the skin surface and in the pilosebaceous follicle duct (see below). In general, microorganisms interact with a family of skin proteins known as extracellular matrix (ECM)-components:
plasminogen, fibronectin, laminin or mucin. For gram-positive bacteria (single membrane), adhesion proteins are used: Bap family adhesins, type 4 pili (important for early biofilm formation), SAATs (Self-associating autotransporters, promoting aggregation between bacteria carrying SAATs), intimins/invasins, etc.; and for gram-negative bacteria (double membrane—LPS): Bap family adhesins displaying an LPXTG C-terminal domain, IMPs (Inner-membrane proteins), type 3 and 4 pili, etc.

One example is Cutibacterium acnes, a commensal bacterium that can become an opportunistic pathogen and cause acne. This bacterium can form a biofilm either on pilosebaceous follicles or on keratinocytes, by virtue of proteins present on the surface of the bacterial membrane, such as CAMP factors (Christie-Atkins-Munch-Petersen factors), sialidases, dermatan-sulphate adhesins, endoglycoceramidases, groEL chaperonins, an “SH3 domain-containing lipoprotein,” a pili/fimbriae-like protein Flp, and above all a DsA1 protein, forming part of the family of MSCRAMMs (molecular surface components recognizing adhesive molecules of the matrix), which is highly immunogenic and provokes a strong immune response in acne sufferers.

The bacterial genus Staphylococcus plays a major role in the skin microbiota with Staphylococcus epidermidis, which is a commensal and rarely pathogenic species, and Staphylococcus aureus, a commensal species with a healthy nasal carriage in 30% of the population, which can become pathogenic, particularly in cases of atopic dermatitis or eczema. The genus Staphylococcus also uses MSCRAMMs such as proteins from the Clf-Sdr family, Bbp (bone sialoprotein-binding protein), FnBPs (fibronectin-binding proteins) and CNA (collagen adhesion) to form biofilms on the skin, and the SesJ protein has recently been identified for S. epidermidis (Arora 2020). For S. aureus, aureusimine (phevalin) appears to be a marker of the biofilm phenotype. Note that the Staphylococcus genus also secretes a polysaccharide intercellular adhesin (PIA) extracellular matrix. The other main representatives of the skin microbiota are (Byrd et al., 2018) either common to all skin types: Corynebacterium tuberculostearicum, Malassezia Globosa; or more characteristic of oily, or dry or moist skin: Staphylococcus capitis, Staphylococcus hominis, Streptococcus mitis, Streptococcus oralis, Micrococcus luteus, Corynebacterium simulans; or microorganisms characteristic of older skin: Dermococcus, Actinomyces;young skin with Bacteroidetes such as: Bacteroides, Alistipes, Prevotella, Porphyromonas, Sphingobacterium, or with Firmicutes such as: Lactobacillus, Aerococcus, Oscillospira, Ruminococcus.

Organisms often considered non-pathogenic can cause infection in hosts with weakened immunity or who have recently received an antimicrobial treatment. When immune responses are impaired, as is often the case with people with diabetes, they may not prevent colonization by pathogenic bacteria in the injured tissue. In chronically infected wounds, many bacteria form a biofilm, wherein they irreversibly attach and grow on a surface, produce extracellular polymers that facilitate matrix formation and modify their phenotype.

The most common bacteria found on human skin are Gram-positive and mainly belong to 5 genera. The following bacteria are found:
Staphylococcus, often opportunistic like Staphylococcus epidermidis, which accounts for over 90% of the aerobic resident flora present on the stratum corneum. Other staphylococci that have been found in skin biofilm are Staphylococcus aureus (common, found, for example, in asymptomatic carriage in nasal cavities in 30% of cases) and Staphylococcus hominis;
Corynebacterium
Propionibacterium (for example, Cutibacterium acnes, Propionibacterium granulosum, Propionibacterium avidum). Cutibacterium acnesproduces fatty acids from the lipolysis of sebum. In doing so, it acidifies the skin environment, which inhibits the growth of colonies of Streptococcus pyogenes;
Lactobacillus;
Streptococcus.

Generally, 3 bacteria are encountered in the majority of cases: 1) Staphylococcus epidermidis, a commensal skin bacterium with a barrier role against Staphylococcus aureus; 2) Cutibacterium acnes, a commensal skin bacterium that is generally harmless, but can cause episodes of acne depending on age (puberty), hormonal impregnation, immunodepression (transient, iatrogenic, etc.); 3) Staphylococcus aureus, a commensal bacterium carried asymptomatically by 30% of individuals (nasal carriage), causing various skin pathologies (impetigo, atopic dermatitis, etc.).

To give an overview of the field of the present disclosure, it can be said that skin diseases are largely forgotten in public health as they are rarely life-threatening. Yet they affect the quality of life of 16 million French people, with psychological repercussions that are often underestimated, linked to skin imperfections (spots, redness, pimples, etc.) that sufferers will attenuate or mask by using cosmetic products.

In France, 1 in 3 people suffer from a skin disease, according to a vast epidemiological study (the “Objectifs Peau” study carried out in 2016 on a representative sample of 20,012 people aged over 15) conducted by the French Dermatology Society (SFD). And 80% of the patients concerned even have two skin diseases. And women are more affected than men: 33% of women have a dermatological pathology, compared to 28% of men.

These alarming figures are far higher than previous estimates. The stresses of modern life and increasing pollution certainly play a significant role in the increased number of skin disorders.

Among the most common skin conditions, acne tops the list (3.3 million French people suffer from it even after puberty), followed by eczema (2.5 million sufferers) and psoriasis (2.4 million). Scalp diseases (excluding alopecia), mycoses and nail diseases come in second, with 2.3 million, 2.2 million and 2.1 million French sufferers, respectively.

For all these conditions, there is a combination of individual criteria affecting the condition of the skin (genetic susceptibilities—mutations in genes coding for skin proteins such as filaggrin, defective immune system, etc.,—, lifestyle—stress, UV booths, etc.,—, diet, etc.) and criteria affecting the microbiota (Cesarean section, hygiene, environment, etc.). Concerning the effect of UV radiation, a pilot study (Burns, 2019) shows an increase in the phylum Cyanobacteria, a decrease in the families Lactobacilluseae and Pseudomonadaceae, a reactivity of the different species according to UVA and UVB, and a potential protective and anti-inflammatory role of Lactobacilluseae. Here too, a test to assess the effect of UV exposure on the human-microbiota symbiosis could help improve advice, on the one hand, in terms of products that rehydrate the skin and regenerate/rebalance the microbiota (curative advice), and on the other hand, in terms of protective sun creams (preventive advice).

This is why cosmetics companies have for some years been interested in understanding the skin microbiota in order to offer their customers suitable and customized compositions that enable them to prepare active creams that are both suited to the physiology of the skin (oily, dry, irritated, with a certain level of inflammation, etc.) and also best suited to the state of the skin microbiota of each customer, at a given period since it has become clear that the claimed effects of anti-wrinkle and anti-aging creams and of various cosmetic skin care products are not similar in their effects and efficacy on all individuals, or even, for the same individual, for all periods. Different people and different skin types react differently to cosmetic products, hence the need for devices that can determine the effects or the reactivity of an individual to a particular type of skin care product. This requires a reliable and simultaneous method for analyzing skin condition and skin microbiota.

### The Skin

The second member of the symbiosis concerned by the field of the present disclosure is the “skin” organ: the exposed part of the skin is called the horny layer (stratum corneum), a superposition of anucleate and completely keratinized cells, the corneocytes, forming highly elongated lamellae (ROBERT et al. Dermopharmacologie, Edisem, 1985). Its thickness is around 10 um except on the palms of the hands and on the soles of the feet where it is about 10 times thicker.

This horny layer is the final external product of this organ, the skin, covering an average of 1.5 to 2 m². The skin is structured into 3 layers of tissue: the hypodermis (deepest), the dermis (intermediate) and the epidermis (most superficial).

The hypodermis forms the deepest layer of the skin. It is a richly vascularized connective tissue that contains a lot of adipose tissue, itself made up of cells called adipocytes. This layer is highly elastic, capable of absorbing shocks and, moreover, insulating the body thermally.

The dermis is the intermediate layer. It is also a connective tissue that supports the epidermis. It is crisscrossed by a rich network of capillaries and is swarming with numerous nerve endings. It is divided into two: the papillary dermis (superficial) and the reticular dermis (deep and medium). Fibroblasts scattered throughout the dermal layer synthesize collagen fibers and elastic fibers, which are immersed in a kind of gel called the extracellular matrix made up of water and glycoproteins. This plays the role of a water reservoir, which can be affected by the environment (dry weather in winter), or overexposure to ultra-violet radiation (sunbathing, UV booths, etc.) leading to increased evaporation. Immune system cells such as macrophages, dermal dendritic cells, mast cells, lymphocytes and maturing cells are also present. The rich vascularization of this layer supports several functions. It enables the body's first line of defense to respond effectively to any danger signal, in particular, with the microbiota in the event of dysbiosis or when a wound creates a breach in the protective upper outer layer. The epidermis, devoid of capillaries, draws the energy and nutrients necessary from them to ensure its cellular activity. Alongside the sweat glands, it plays an important role in thermoregulation. The sweat glands and pilosebaceous follicles are epidermal appendages implanted in the dermis.

The epidermis is the outermost, non-vascularized structure, divided into 5 superimposed layers (internal to external):
The basal layer (stratum basale) enables the skin to regenerate by virtue of cell divisions (almost all cells are in mitosis) which allow the cells produced to migrate toward the outermost layer. This layer is composed mainly of keratinocytes.
The spiny layer (stratum spinosum or Malpighian layer) is the thickest layer of the epidermis. It is composed of keratinocytes.
The thinner granular layer (stratum granulosum) is made up of keratinocytes undergoing apoptosis. Their cytoplasm comprises fewer cytoplasmic organelles and less nuclear chromatin. The cells are flatter and characterized by the presence of keratohyalin granules and lamellar granules (or Odland bodies).
The clear layer (stratum lucidum) has anucleate cells (their nuclei have disappeared). The keratohyalin granules transform into a protein called filaggrin.
The horny layer (stratum corneum), the most superficial layer, is made up of flattened, dead cells known as corneocytes. They are said to be dead but they remain biologically active. They have lost all their organelles, replaced by dense keratin filaments and are bound together by an interlipid cement of fatty acids, cholesterol and ceramides as well as corneodesmosomes. This cement is formed from the Odland bodies found in the preceding layers. These tightly-packed connected cells form an impermeable coating on the skin and play an important defensive role.

The keratinization process ensures continuous renewal of the skin, from the basal layer up to the horny layer, within 3-4 weeks. The various constituent elements will be found on the surface and will constitute the surface of the stratum corneum on which the microbiota forms a biofilm. Biological markers of the condition of the skin will be found here making it possible to assess whether it is in a healthy state, hormonal impregnation (puberty, menstrual cycle, etc.), inflammation (endogenous or exogenous), an allergic reaction, aggression (physical: ultra-violet radiation, pollution; chemical: personal care products, cosmetics; mechanical: abrasion during scrubbing, exfoliation or peeling, etc.).

The stratum corneum is the first line of defense between the human body and the outside world, acting as a barrier against the penetration of external agents, preventing the passage of deleterious microbial or chemical agents into the body. Should this first level of protection fail, another protective mechanism is brought into play involving the cells of the immune system.

The stratum corneum is the first level of the interactome, in direct contact, where the microbiota forms a biofilm using adhesion proteins and secretes exopolysaccharides enabling it to persist on the skin.

When the physical barrier of the stratum corneum no longer operates because it has been physically removed by abrasion, for example, on the surface following a pronounced exfoliation or peeling, or more deeply in the case of a traumatic or surgical wound, it is the skin's immune system that comes into play. Its purpose is to protect the host and if necessary to restore the integrity of the skin. It is divided into two types: firstly innate immunity, then adaptive or specific immunity. In both cases, the first guardians are the keratinocytes as they are the most numerous (90% of skin cells). They act as immune sentinels and recognize foreign agents by virtue of pattern recognition receptors (PRR) from the Toll-like receptor family, which, once recognized, synthesize chemical mediators (cytokines and chemokines). They initiate the inflammatory cascade. On the other hand, keratinocytes are also capable of producing antimicrobial peptides, which inhibit microorganisms such as Staphylococcus aureus and Candida albicans.

The epidermis contains 2 types of immune cells. Dendritic cells, or Langerhans cells, with pseudopods that enable them to latch on to the pathogen and phagocytose it. These cells are known as APCs: antigen-presenting cells. Once the pathogen or antigen has been phagocytosed, the dendritic cell secretes chemical mediators (prostaglandins or chemokines) which cause local vasodilatation and therefore increase the blood flow and the local recruitment of other immune cells such as polymorphonuclear neutrophils and macrophages. Dermal immune cells (dermal dendritic cells, macrophages, mast cells) are also mobilized at the site concerned. The epidermis also harbors T lymphocytes that have been activated in the lymph nodes by virtue of APCs. Thus, after the activation of APCs, a cascade of multiplication and differentiation ensues, leading ultimately to the destruction of pathogens. These biological markers can be used to assess the condition of the skin surface, in particular, the level of inflammation, which may be chronic and becomes increasingly critical with age, known as “inflammaging.”

Comprehensively, biological markers of skin inflammation include interleukins IL-1beta, IL-4, IL-6, IL-8, IL-11, IL-12, Tumor Necrosis Factor-alpha (TNF-alpha), Interferon-gamma (IFN-gamma), Granulocyte Macrophage Colony-Stimulating Factor (GM-CSF), Transforming Growth Factor-beta (TGF-beta). Although not directly considered a marker of inflammation, mention must also be made of Immunoglobulin E (IgE), whose serum level is elevated in cases of atopic eczema-dermatitis and is responsible for the majority of so-called IgE-dependent allergic reactions.

Other skin cells are involved in maintaining a healthy state in the face of the sun's aggression or exposure to ultra-violet radiation (for example, in booths using UV lamps). These include melanocytes present in the skin, which play a protective role against ultraviolet (UV) radiation. When the skin is exposed to UV light, keratinocytes secrete melanotropic hormone or α-MSH (α-melanocyte stimulating hormone) by maturing a prohormone, POMC (proopiomelanocortin). This α-MSH hormone attaches to a membrane receptor called MCR (melanocortin receptor) expressed by melanocytes and triggers melanin biosynthesis, which is the source of skin tanning. This thus protects the genetic material of epidermal cells exposed to UV radiation.

The description of the “skin” organ is completed with the appendages included within the different layers: hair and the sebaceous and sweat glands.

Hair covers almost the entire human body with varying sizes. They play a very important role in thermoregulation. They are anchored in the dermis in what is called the hair follicle.

The sebaceous gland attached to the hair is an intradermal gland. It synthesizes sebum, a major component along with sweat of the hydrolipidic film that protects the skin and prevents it from drying out. This gland interacts with the immune system with a subsequent impact on the microbiota as will be seen later.

The sweat gland secretes sweat and, through this process, enables the body to combat heat. In fact, when body temperature rises, for example, during a sports session or when you are ill, the sweat released evaporates at the surface of the skin: this mechanism eliminates heat and contributes, along with vasodilation, to cooling the body.

Of these appendages, the sebaceous glands play a central role conditioning the pH of the skin, its more or less occlusive character and therefore access to oxygen (impact on the aerobic or anaerobic capacities of the microorganisms of the microbiota), antioxidant effects, pro- and/or antimicrobial effects (variable depending on the microorganism species), and the transport of hormones/pheromones. Sebum is composed of triglycerides, diglycerides, free fatty acids, wax esters, squalenes and cholesterols.

### The Skin-Microbiota Symbiosis

The human-microbiota symbiosis at the skin level is all the more complex as it has recently been disclosed that so-called “innate lymphoid” immune cells (ILCs) play a regulatory role on sebaceous glands (Kobayashi et al., 2019). These ILCs (by producing “TNF receptor ligands”) limit the growth of sebaceous glands. When these ILCs are deficient, sebaceous gland hyperplasia is observed resulting in increased production of antimicrobial lipids, particularly palmitoleic acid and oleic acid, with an impact on the balance of the skin microbiota. Indeed, palmitoleic acid inhibits the growth of gram-positive aerobic cocci such as Staphylococcus aureus and Staphylococcus xylosus, but not the growth of gram-negative anaerobes such as the Bacteroides species. Combined with the overproduction of oleic acid, this creates an occlusive film, that is, anaerobic conditions unfavorable to gram-positive cocci. This may lead to cosmetic products containing these two fatty acids not being recommended in cases where biological markers correlated with the “oily skin+high presence of Staphylococcus” profile have been characterized with the immunochromatographic revealing device forming the subject of the present patent.

The human-microbiota symbiosis is also organized at levels other than that of the epidermis-stratum corneum but also, more broadly, based on various endogenous factors such as body region (T-zone of the face, scalp, armpits, etc.), age, hormonal impregnation (puberty, menstrual cycle, etc.), inflammation (of endogenous origin—atopic dermatitis, psoriasis etc.—or exogenous—contact with an irritating product—), aggression (physical: ultra-violet radiation, pollution; chemical: personal care products, cosmetics; mechanical: abrasion during exfoliation or peeling, etc.).

Body regions are segmented into three zones characterized by different physicochemical parameters:
Moist Zones:
The so-called “moist” zones are characterized by a high density of sweat glands. The moisture is due to the secretion of sweat by these glands. Sweat is composed mainly of water, mineral salts, uric acid and urea. The zones of the human body concerned are: the axilla, the perineum, the interdigital folds, the palms of the hands and the armpits where there is a high level of colonization by micro-organisms: 105-108 bacteria/cm².
Lipid Zones:
The so-called “lipid” zones are associated with a high presence of sebaceous glands. These glands secrete sebum, which flows to the surface of the skin in the form of a lipid film. The head, notably the cheeks, forehead, nose, chin, trunk and upper back, are the main lipid zones with a concentration of micro-organisms of the order of 106-107 bacteria/cm².
Dry Zones:

The so-called “dry” zones are poor in sweat and sebaceous glands and therefore contain less sweat and sebum secretions.

The back of the hands and the outside of the limbs are the main dry zones. These have the lowest concentration of micro-organisms: 10³-10⁴ bacteria/cm².

The average pH of the skin is 5.5. It is due to the hydrolipidic film that covers the entire surface of the skin, produced by secretions from the sweat glands and sebaceous glands. The relative acidity of this film notably provides protection against pathogens. Skin pH is a very important parameter to maintain for skin homeostasis and the balance of the biofilm microbiota. In the course of numerous pathologies, such as atopic eczema—dermatitis, psoriasis, acne, etc., the pH is altered and it has been documented to be altered in cases of atopic dermatitis correlated with the presence of Staphylococcus aureus (Rippke et al., 2004).

### Solution Provided by the Present Disclosure

The present disclosure remedies the disadvantages of the background art by offering a solution:
Fast;
Inexpensive;
Comprising sufficient elements to characterize the skin; and
Allowing several levels of biomarker readings to give sufficient information to make a cosmetic recommendation.

Thus, the present disclosure relates to a device for immunochromatographically revealing the skin-microbiota complex comprising a support on which is arranged a transfer well intended to receive a solution comprising the constituents of the skin-microbiota complex, the well leading to a plurality of N revealing zones each having (i) an immunological detection reagent and (ii) an immunological capture reagent, and the skin-microbiota complex consisting of skin cells and microorganisms and other constituent elements of the skin microbiota, the device comprising:
a number of revealing zones N, where N is equal to or greater than 5;
at least one immunological detection reagent specific for a skin cell biomarker and at least one immunological detection reagent specific to a biomarker of the skin microbiota;
the immunological detection reagents each comprise an antibody specific to a biomarker of skin cells or of the skin microbiota, conjugated to a colorimetric identification system;
the capture reagents each comprise an antibody, bound to the support in each of the N revealing zones, capable of recognizing the biomarker present in the zone; and
at least one fool-proofing means for orienting the reading of the device,
   characterized in that it comprises:
at least three zones for revealing three biomarkers of the skin microbiota, the biomarkers being bacteria from each of the genera Staphylococcus, Cutibacterium and Corynebacterium; and
at least one zone for revealing a skin cell biomarker selected from (i) a structural protein, (ii) an inflammatory biomarker or (iii) an allergy biomarker.

It also relates to a single-use kit for characterizing the condition of the skin, a system for implementing a personalized cosmetic recommendation and a personalized cosmetic recommendation method based on characterizing the skin-microbiota complex of an individual and implementing the device according to the present disclosure.

Just as the intestinal microbiota interacts with the digestive tract in a “microbiota-host” symbiosis, the skin microbiota also interacts with the skin in a symbiotic relationship. Similarly, there are as many types of skin as there are microbiota, and damage to one member of the symbiosis has repercussions for the other. This is important for maintaining healthy skin or for correcting imperfections with cosmetic care products. The present disclosure enables a rapid and significant biological determination of the two members of the symbiosis by virtue of immunochromatographic characterization of biological markers accessible on the surface of the microbiota-skin interactome. Artificial intelligence is then used to recommend the best care suited, on the one hand, to the skin type and compatible, on the other hand, with the microbiota thereof, thereby reducing the risk of adverse effects. Similarly, in the event of skin imperfections and/or microbiota imbalance, the present disclosure recommends the best corrective treatment that is either guaranteed to be compatible with the microbiota or capable of regulating same.

### BRIEF DESCRIPTION OF THE DRAWINGS

FIG. 1: Depiction of an embodiment of a device for immunochromatographically revealing the skin-microbiota complex according to the present disclosure, comprising a support (0) on which is arranged a transfer well intended to receive a solution comprising the constituents of the skin-microbiota complex, the well leading to a plurality of N revealing zones. (1) Sampling device (swab, cotton bud, etc.), (2) Sampled solution from the skin, (3) Transfer well, (4) Absorbent zone of sampled solution, (5) Zone with detection conjugate (or monoclonal detection antibody), (6) Monoclonal detection antibody, (7) Direction of migration flow, (8) Zone on which a monoclonal capture antibody has been deposited, (9) Monoclonal capture antibody (or antibody fragment, or nanobody), (10) Control zone, (11) Antibody (or antibody fragment, or nanobody) directed against the conjugate.

FIG. 2: Depiction of 3 embodiments of detection strips that can be used in a device for immunochromatographically revealing the skin-microbiota complex according to the present disclosure. (A) 1 biomarker detected with several detection thresholds (for example, for one microorganism: threshold 1=10³ CFU; threshold 2=10⁵ CFU; threshold 3=10⁷ CFU; threshold 4=10⁹ CFU; for one molecule: threshold 1=1 ng; threshold 2=10 ng; threshold 3=100 ng; threshold 4=1000 ng); the Control corresponds to the detection by an antibody (or antibody fragment) (B) 3 microbiota biomarkers detected over 4 zones; the Control corresponds to the detection by an antibody (or antibody fragment) (C) 2 skin biomarkers detected over 4 zones; the Controls correspond to biomarker deposits (validation of the migration of the detection conjugate specific to each biomarker). The intensity of the bands shows the fact that the control zones must be saturated, while the intensity of the other zones depends on the amount of analyte that has migrated, thus enabling semi-quantitative analysis of biomarkers.

FIG. 3: Depiction of an embodiment of a support (0) for a device for immunochromatographically revealing the skin-microbiota complex according to the present disclosure, on which is arranged a (central) transfer well (3) intended to receive a solution comprising the constituents of the skin-microbiota complex, the well leading to a plurality of N revealing zones arranged on strips radially arranged around the central transfer well (herein 8 strips for illustration purposes, each comprising 4 or 5 revealing zones). This support comprises at least one fool-proofing means for orienting the reading of the device (12), materialized by a black triangle, which can be, for example, a notch or a pattern and allows the reading direction of the support to be oriented.

FIG. 4: Schematic illustration of the process leading to a personalized cosmetic recommendation. (A) depiction of the semi-quantitative analysis of the microbiota profile, the same type of analysis being carried out for one or more skin biomarkers (B) presentation of the result of the combinatorial analysis of biomarkers to establish the characterization of the skin-microbiota complex (C) recommendation of suitable cosmetic products based on a product database and related to the state of the skin-microbiota complex.

### DETAILED DESCRIPTION

A first object of the present disclosure relates to a device for immunochromatographically revealing the skin-microbiota complex comprising a support on which is arranged a transfer well intended to receive a solution comprising the constituents of the skin-microbiota complex, the well leading to a plurality of N revealing zones each having (i) an immunological detection reagent and (ii) an immunological capture reagent, and the skin-microbiota complex consisting of skin cells and microorganisms and other constituent elements of the skin microbiota, the device comprising:
a number of revealing zones N, where N is equal to or greater than 5;
at least one immunological detection reagent specific for a skin cell biomarker and at least one immunological detection reagent specific to a biomarker of the skin microbiota;
the immunological detection reagents each comprise an antibody specific to a biomarker of skin cells or of the skin microbiota, conjugated to a colorimetric identification system;
the capture reagents each comprise an antibody, bound to the support in each of the N revealing zones, capable of recognizing the biomarker present in the zone; and
at least one fool-proofing means for orienting the reading of the device,
characterized in that it comprises:
three zones for revealing three biomarkers of the skin microbiota, the biomarkers being three bacteria of the genera Staphylococcus, Cutibacterium and Corynebacterium, respectively; and
a zone for revealing a skin cell biomarker selected from (i) a structural protein, (ii) an inflammation biomarker or (iii) an allergy biomarker.

For the purposes of the present disclosure, the “skin-microbiota complex” is made up of two cell types: skin cells of human origin and microorganisms; these two cell types participate in the condition of the skin by interacting with each other. Phenomena of symbiosis are observed. Thus, by simultaneously sampling human cells and the microbiota present on the surface of the skin, there is a representative sample of a skin condition. This complex also contains other constituent elements of the skin-microbiota complex that are secreted by the two types of cells. On the one hand, molecules secreted by the skin cells, whether epidermal or immune, but also secretions from the microorganisms that make up the microbiota.

The aim herein is to establish a holistic characterization of the skin condition based on revealing biomarkers representative of the physiological state of the skin and of the constitution of the microbiota to enable their combinatorial analysis. The number of revealing zones N corresponds to the number of biomarkers tested. N is greater than 5 so that a sufficient number of biomarkers can be tested to obtain a value-added result resulting from the combinatorial analysis of the different biomarkers. The device also comprises at least one control zone, not included in the “at least 5 zones.”

The “at least 5 zones” of the device reveal at least one skin cell biomarker selected from (i) a structural protein, (ii) an inflammation biomarker or (iii) an allergy biomarker and at least three biomarkers of the skin microbiota, these three biomarkers being bacteria belonging to the genera Staphylococcus, Cutibacterium and Corynebacterium.Preferred embodiments are described below.

In particular embodiments of the present disclosure, at least 8, 10, 15, 20, 25, 30 different types of biomarker are tested simultaneously.

The device not only reveals the absence or the presence of the biomarkers considered but also quantifies them in absolute or relative terms, based on the method selected.

### The Microbiota

The skin microbiota constitutes the first element of the symbiotic complex revealed by the device according to the present disclosure. It comprises a range of microorganisms including bacteria, yeasts, fungi and mites.

In order to provide information about the state of the skin microbiota, the device according to the present disclosure can at least reveal the presence of bacteria of the genera Staphylococcus, Cutibacterium and Corynebacterium.

Preferably, the species of Staphylococcus investigated will be selected from Staphylococcus epidermidis and Staphylococcus aureus. These biomarkers can be revealed either via an antibody capable of recognizing at least these two species of the genus Staphylococcus (genus-specific antibody), or via two antibodies specific to the species Staphylococcus epidermidis and Staphylococcus aureus, respectively.

Preferably, the species of Cutibacterium investigated will be selected from Cutibacterium acnes and Cutibacterium granulosum. These biomarkers can be revealed either via an antibody capable of recognizing at least these two species of the genus Cutibacterium (genus-specific antibody), or via two antibodies specific to the species Cutibacterium acnes and Cutibacterium granulosum, respectively.

Preferably, the species of Corynebacterium investigated is selected from Corynebacterium xerosis and Corynebacterium kroppenstedtii.These biomarkers can be revealed either via an antibody capable of recognizing at least these two species of the genus Corynebacterium (genus-specific antibody), or via two antibodies specific to the species Corynebacterium xerosis and Corynebacterium kroppenstedtii, respectively (species-specific antibody).

Within the context of the present disclosure, the skin microbiota is analyzed by revealing epitopes exposed on the surface of the microbiota. In a particular embodiment of the present disclosure, the epitope exposed on the surface of the microbiota is associated with a protein present on the surface of a microorganism constituting the microbiota. In particular, it may be an epitope associated with a biofilm-like behavior of the microbiota.

The revealing zones can be used to reveal “additional biomarkers of skin microbiota,” that is, biomarkers other than those that reveal the presence of bacteria belonging to the genera Staphylococcus, Cutibacterium or Corynebacterium or to the associated species mentioned previously. These other biomarkers can reveal the presence of a microorganism selected from bacteria, yeast, fungi or mites.

Preferably, the microorganisms making up the microbiota that will be investigated in order to establish a diagnosis of the skin condition within the scope of the present disclosure are selected from the following bacteria (Byrd et al., 2018):
Staphylococcus epidermidis, Staphylococcus aureus, Staphylococcus lugdunensis, Staphylococcus hominis (H, G), Streptococcus mitis (H, G), Streptococcus oralis(S), Streptococcus pseudopneumoniae (S), Streptococcus sanguinis(S), Staphylococcus capitis, Cutibacterium acnes, Corynebacterium simulans, Corynebacterium fastidiosum (H), Corynebacterium afermentans (H), Corynebacterium xerosis, Corynebacterium aurimucosum (G), Corynebacterium kroppenstedtii (G), Corynebacterium amycolatum (G), Corynebacterium tuberculostearicum,
Veillonella parvula(S),
Micrococcus luteus (S, H),
Enhydrobacter aerosaccus (H),
Epidermophyton floccosum,
Nannizzia nana,
Nephroselmis olivacea,
Cyanophora paradoxa,
Aureoumbra lagunensis,
Pycnococcus provasolii Pyramimonas parkeae,
Parachlorella kessleri,
Aspergillus tubingensis,
Zymoseptoria tritici,
Tilletia walkeri,
Or bacteria of the genera Dermacoccus, Actinomyces, Bacteroides, Alistipes, Prevotella, Porphyromonas, Sphingobacterium, Lactobacillus, Aerococcus, Oscillospira, Ruminococcus.

Other microorganisms making up the skin microbiota that can be investigated in order to establish a diagnosis according to the present disclosure are:
yeasts such as Malassezia Globosa, Malassezia restricta, Malassezia furfur, Malassezia sympodialis, Candida parapsilosis, etc. ;
fungi such as Aspergillus tubingensis, Zymoseptoria tritici, Tilletia walkeri, Epidermophyton floccosum, Nannizzia nana, Nephroselmis olivacea, Cyanophora paradoxa, Aureoumbra lagunensis, Pycnococcus provasolii, etc. ; and
mites such as Demodex folliculorum and Demodex brevis.

Thus, the device according to the present disclosure makes it possible to analyze the presence of microorganisms, optionally to characterize their organization in the form of biofilm, but also to analyze the presence within the complex of other elements whose presence provides interesting information about the condition of the skin. When these other elements are immunogenic, it is possible to provide the device with antibodies capable of revealing their presence.

By “epitopes exposed on the surface of the microbiota,” this means epitopes exposed on the surface of the microorganisms making up the microbiota via the molecules making up the membrane of the microorganisms such as proteins, complex fatty acids and polysaccharides, but also epitopes formed by secreted molecules such as exopolysaccharides making up the biofilm matrix.

The skin cells harvested on the surface are cells of the stratum corneum or depending on the intensity of the sampling (abrasion) or on the condition of the skin (exposed after scrubbing or intense exfoliation or peeling), cells from the underlying stratum lucida or even stratum granulosum, which are even more deeply buried under normal circumstances.

By “proteins present on the surface of microorganisms,” this means proteins exposed on the membrane: secreted proteins and structural proteins synthesized by cells that transit or are attached to the cell membrane, as in the case of secreted proteins. By way of illustration, Staphylococcus aureus produces the Panton-Valentine Leukocidin protein, and an extracellular matrix “polysaccharide intercellular adhesin” (PIA), Cutibacterium acnes produces porphyrins, Pseudomonas an alginate-based matrix, Escherichia coli a colanic acid-based matrix, Cutibacterium a β-1,6-linked N-acetylglucosamine (PNAG)-based matrix; for structural or adhesion proteins on the skin surface: for gram-positive bacteria (single membrane): Bap family adhesins, type 4 pili (important for early biofilm formation), SAATs (Self-associating autotransporters, promoting aggregation between bacteria carrying SAATs), intimins/invasins, etc. ; more specifically, for Cutibacterium acnes, proteins present on the surface of the bacterial membrane, such as CAMP factors (Christie-Atkins-Munch-Petersen factors), sialidases, dermatan-sulphate adhesins, endoglycoceramidases, GroEL chaperonins, an SH3 domain-containing lipoprotein, a Flp pili/fimbriae-type protein, and above all a DsA1 protein; more specifically, for the bacterial genus Staphylococcus, MSCRAMMs (molecular surface components recognizing adhesive molecules of the matrix) such as proteins of the Clf-Sdr family, with Bbp (bone sialoprotein-binding protein), FnBPs (fibronectin-binding proteins), and CNA (collagen adhesion), the SesJ protein has recently been identified for S. epidermidis (Arora et al., 2020), while for S. aureus, aureusimine (phevalin) seems to be a biofilm phenotype marker; while for gram-negative bacteria (double membrane—LPS): adhesins of the Bap family exhibiting an LPXTG C-terminal domain, IMPs (Inner-membrane proteins), type 3 and 4 pili, etc.

Thus, the proteins exposed on the membrane of the microorganisms making up the microbiota are selected from Panton-Valentine Leukocidin, porphyrins, alginates, β-1,6-linked N-acetylglucosamine (PNAG), Bap family adhesins, type 4 pili, self-associating autotransporters, intimins/invasin, CAMP factors, sialidases, dermatan-sulphate adhesins, endoglycoceramidases, GroEL chaperonins, SH3 domain-containing lipoprotein, Flp pili/fimbriae protein, DsA1 protein; Clf-Sdr family proteins, such as Bbp, FnBPs and CNA, SesJ protein, aureusimine, Bap family adhesins displaying an LPXTG C-terminal domain, IMPs, type 3 and 4 pili.

For gram-positive bacteria, the complex fatty acids exposed on the membrane of the microorganisms making up the microbiota are selected from teichoic or even lipoteichoic acids, which can contain long chains of ribitol phosphate or of glycerol-3-phosphate; whereas for gram-negative bacteria, they are selected from lipopolysaccharides, which are glycolipids comprising a lipid region called lipid A, which is most commonly made of a disaccharide of phosphorylated glucosamines and containing fatty acids with ester or amide linkages.

The sugars exposed on the membrane of the microorganisms making up the microbiota are selected from glycopolymers (for example, based on rhamnans) and peptidoglycans.

In a preferred embodiment of the present disclosure, at least one of the skin microbiota-specific biomarkers corresponds to a biomarker associated with a biofilm-like behavior of the microbiota. Bacteria and other microorganisms in biofilm form are defined as opposed to bacteria and other planktonic microorganisms, which are mobile and non-adherent. The biofilm-specific biomarker may correspond to a protein present on the surface of the membranes of the microorganisms or to another element of the microbiota.

When the presence of a bacterium or other microorganism in biofilm form is investigated, the biomarkers are selected from adhesion proteins such as fimbriae, curli, pili, etc., which are characteristic of the presence of the microbiota biofilm naturally formed on the skin.

### The Skin

The condition of the skin constitutes the second element of the symbiotic complex revealed by the device according to the present disclosure. The skin condition is analyzed by virtue of at least one skin cell biomarker selected from (i) a structural protein, (ii) an inflammation biomarker or (iii) an allergy biomarker.

Structural proteins are targeted when the presence of skin cells are revealed and/or the quality of the skin is assessed. The use of structural protein-like biomarkers can also be used to quantify the number of skin cells present in the sample taken or to normalize the level of biomarkers. The structural proteins of interest include keratin, filaggrin, loricrin, etc.

Inflammatory biomarkers are selected from C-Reactive Protein (CRP), interleukins IL-1beta, IL-4, IL-6, IL-8, IL-11, IL-12, Tumor Necrosis Factor-alpha (TNF-alpha), interferon-gamma (IFN-gamma), Granulocyte Macrophage Colony-Stimulating Factor (GM-CSF), Transforming Growth Factor-beta (TGF-beta).

Allergy biomarkers are selected from immunoglobulins, IgE, IgA and IgG, preferably IgE. Indeed, serum IgE levels are elevated in cases of atopic eczema or dermatitis.

In a preferred embodiment of the present disclosure, the “at least one” skin cell biomarker investigated is IL-1beta. For the other two types of skin cell biomarkers, the structural protein is preferably keratin, and the allergy biomarker is preferably IgE.

In a preferred embodiment of the present disclosure, the device comprises zones for specifically revealing the presence of bacteria of the genera Staphylococcus, Cutibacterium and Corynebacterium and IL-1beta. In a more particular embodiment, the device further comprises a zone for revealing the presence of IgE. In an even more particular embodiment, it further comprises a zone for revealing the presence of keratin.

### The Device as a Biomarker Support

The single-use immunochromatographic revealing device is sterile-packed and dehydrated. This module takes the form of a support, typically made of plastic, having a well for the solubilization solution containing the harvested biological material, leading to a series of channels (for example, nitrocellulose strips or lateral flow strips) containing a plurality of recognition and revealing zones specific to a biomarker of interest. These channels may, for example, receive antibody-bearing nitrocellulose strips, wherein the solubilization solution containing the harvested biological material migrates by capillary action on the strip.

In a preferred embodiment, the device according to the present disclosure is a lateral flow immunochromatographic device.

Detection antibodies can be conjugated either to a nano-sized gold particle (allowing only one red marking color per strip, but different capture lines), or to a colored particle (multiplying the number of detections per strip), or, for example, latex (blue color).

Several different configurations of revealing zones are possible depending on the biomarker revealing mode.

The revealing zone can be set up on a nitrocellulose strip with a single uniform concentration of capture antibody over the entire width of the strip (homogeneous zone) to form a line revealed by interaction with the analyte (made visible by the detection antibody-conjugate). Several detection zones for different analytes can be combined on one strip, each analyte being detected with its own threshold concentration of capture antibody (FIG. 2). In another arrangement, this revealing zone can also be set up with at least two different concentrations of capture antibody. In alternative embodiments, the zone comprises 2, 3 or more different concentrations of capture antibody.

In addition, it is possible to standardize the detection thresholds of the various biomarkers so that relative quantifications can be established between the different biomarkers. This standardization can be carried out either with respect to a given common marker serving as a reference (for example, keratin), or by establishing the relative quantification of all markers in relation to each other (for example, to bring them to a cumulative total of 100, with each marker obtaining its own pro-rata value).

The device must comprise a fool-proofing means, which is a marking necessary to orientate the device correctly when viewing the signals (the lines visible on the strips) revealing the biomarkers. This fool-proofing means is located directly on the device, on the reading face. It can take a graphic form, such as a drawing, or a physical form, such as a notch, a hole, or any other marking system. It can be made up of one, two or more marking elements.

The device generally consists of a disc, the support for the revealing zones, enclosed in a cassette.

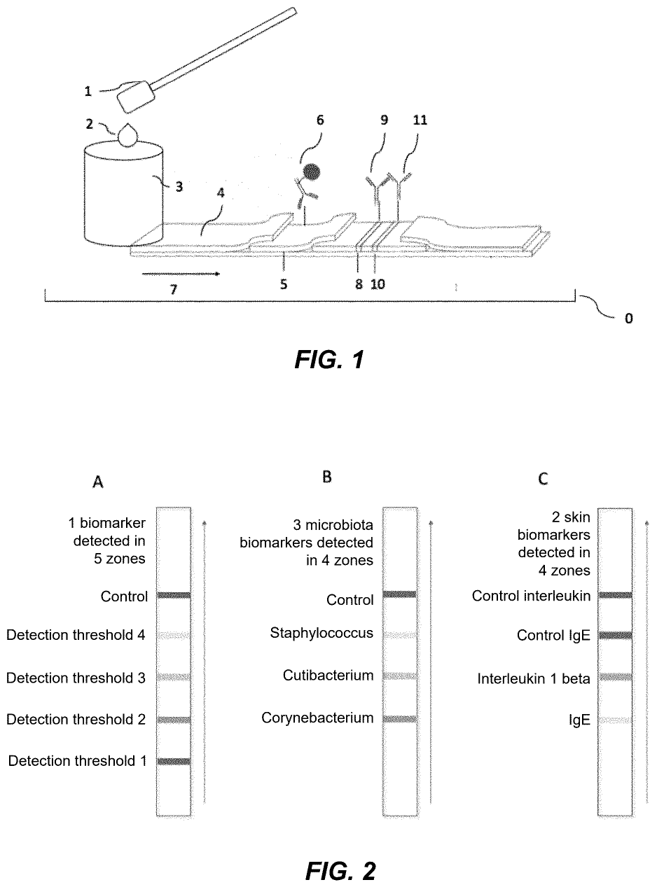

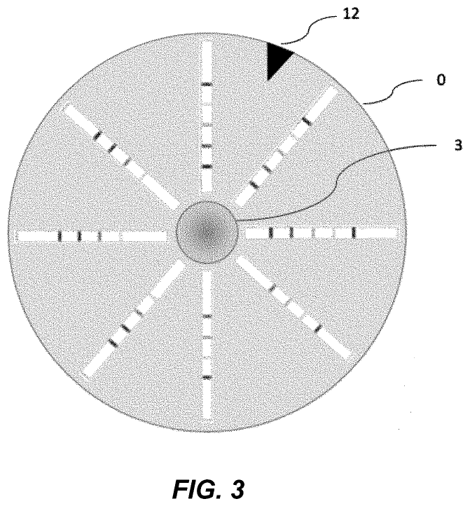

FIGS. 1 to 3 show different embodiments of the device according to the present disclosure.

A second object of the present disclosure relates to a single-use kit for characterizing the skin condition comprising:
A sampling device for the skin-microbiota complex comprising a means of sampling the complex and a receptacle containing a solubilization solution;
A device for immunochromatographically revealing the skin-microbiota complex as described previously.

The sampling means may, for example, consist of a patch, a scraper, a swab soaked in a buffer solution or a sponge soaked in a buffer solution, a tulle or gauze cloth soaked in a buffer solution, or any other suitable means enabling a physical exfoliation, soaking or adsorption action.

The number of microorganisms, notably bacteria, per square centimeter of skin varies greatly according to the skin zone, notably depending on sebum content. Thus, to ensure representative sampling, it is necessary to have a sampling device adapted to the type of skin and the area sampled so as to have sufficient biological material to carry out the analysis of biomarkers of skin cells and of the microbiota. The biological material sampled comprises constituent elements of the exposed part of the skin (cells of the stratum corneum, and/orlucidum, and/or granulosum) and of the microbiota (bacteria, yeasts, fungi, and even mites such as demodex).

Typically, sampling should enable a sufficient number of bacteria to be harvested to obtain a concentration of at least 10³ CFU/mL of bacteria when brought into contact with the solubilization solution intended to receive the harvested biological material. Preferably, for optimum detection, the concentration of the solution containing the harvested microbiota should be 10⁴ CFU/mL of bacteria. However, these concentrations are given for guidance only and depend on the detection sensitivity of the revealing method.

When sampling is performed via a patch, the patch will have an interaction surface (collection surface) with the skin of at least 5 cm², or even 10 cm², 12 cm² or more, depending on the abundance of skin microbiota and on sampling efficacy. The sampling (or harvesting) means is supplemented by a receptacle (such as an extraction tube or equivalent) pre-filled and packaged in sterile packaging, containing a solubilization buffer (typically between 1 mL and 5 mL), for example, a lysis/migration buffer to buffer the pH of the sample, minimize non-specific binding, neutralize interference and control flow rate by virtue of the use of various salts, surfactants, detergents, stabilizing agents or blocking reagents (example composition: PBS 1× with 1% TWEEN® R 20). This receptacle has a volume adapted to directly receive the collection surface of the sampling device so as to dissolve what has been sampled from the surface of the skin.

The single-use kit requires only simple handling that can be carried out immediately after sampling without any biological knowledge and enables a sample to be characterized with respect to a large number of reference biomarkers, to determine combinations representative of a wide variety of skin/microbiota complexes.

A third object of the present disclosure relates to a system for implementing a personalized cosmetic recommendation based on the characterization of the skin-microbiota complex comprising:
A sampling device for the skin-microbiota complex comprising a means of sampling the complex and an extraction tube containing a solubilization solution;
A device for immunochromatographically revealing the skin-microbiota complex as described previously, wherein the N revealing zones constitute zones capable of providing a signal when a biomarker is detected;
A computer performing image analysis processing, the image consisting of all the signals from the N zones revealed by virtue of the immunochromatographic revealing device, to determine the nature of the biomarkers having been recognized in the N revealing zones and categorize the skin condition based on the combination of biomarkers identified.

The computer acquires an image of the various revealing zones after a given reaction time, and transmits this image in digital form to a processing center for automatic analysis in order to characterize the type of biomarkers that have reacted with the antibodies present in the revealing zones of the revealing device.

Images can be acquired in a single shot so as to perform a one-off analysis. This mode of analysis is particularly suited to the use of a device for which the antibody detection thresholds are standardized, or else the revealing zones comprise at least two different antibody concentrations.

Images can also be acquired in several successive shots in order to perform a dynamic analysis. This method makes it possible to monitor the appearance of signals and perform relative quantifications of biomarkers. It can be implemented using a device for which the antibody detection threshold may or may not be standardized. An arrangement can, for example, provide for real-time monitoring of the change in signal intensity (the intensity of the lines in each zone on the strips) to trigger a first shot when the reference biomarker reaches an intensity corresponding to the saturation level (for example, after 5 minutes of migration) and a second shot when the biomarker with the weakest signal reaches an intensity corresponding to the minimum detection threshold (for example, 10 minutes), or when the migration process is deemed to have come to an end (for example, 15 minutes).

The shot can be taken using a Smartphone or equivalent, combined with image analysis software to process it by exploiting the accumulated data (this analysis can involve trained software based on AI-type learning processes comprising both the data provided by the user—age, weight, height, tobacco and alcohol consumption, susceptibility to sugar (diabetes), UV booth sessions, etc.). It is then possible to scan the barcodes of the products used in the cosmetic routine, linking them to information relating to skin biomarkers and microbiota biomarkers, in order to establish a recommendation of products (nutraceuticals, cosmetics, etc.) best suited to the condition of the skin and the microbiota.

These three means form an inseparable whole: biomarker analysis via the immunochromatographic revealing device is only possible because the sampling device allows a sufficient volume of biological material to be collected (skin cells and microbiota).

The use of this kit for a complex analysis due to the large number of biomarkers analyzed on both skin cells and on the skin microbiota, and due to a combinatorial approach corresponding to a large number of arrangements required to categorize the biological material, preventing conclusions from being drawn simply by reading the results as is the case for a Covid test or a pregnancy test, is only possible because of the simplicity of acquiring an image and transmitting same in digital form for processing on a computer that pools the processing of kits for a large number of users.

The channels presenting the detection zones define a plane closed by a transparent window. The user uses this window to take a photograph after the reaction time has elapsed, using, for example, a smartphone running a dedicated application commanding notably:
a) the acquisition of an image;
b) local verification, on the smartphone's processor, of the conformity of the image acquired with respect to test patterns provided on the imaging device, for example;
c) entry of additional information;
d) time-stamping and optional geolocation by the smartphone; and
e) transmission of the image and associated information to a remote

### Image Exploitation

The images received on the server are then automatically processed to recognize detection zones that have reacted to codify the combination of biomarkers present in the analyzed skin-microbiota complex, and to categorize the skin by processing this combination, notably by supervised learning from a reference base of data collected from a panel of people who have been characterized by an expert.

A fourth object of the present disclosure relates to a personalized cosmetic recommendation method comprising the following steps:
Taking a sample of biological material from the surface of the skin using a sampling device;
Suspending skin cells and microorganisms constituting the biological material taken in a solubilization solution;
Pouring the solubilization solution into the transfer well of an immunological revealing device as defined previously;
Incubating between 1 and 10 min in order to allow the cells and microorganisms to react with the reagents on each of the N zones of the immunological revealing device;
Processing the image consisting of the set of signals revealed on each of the N zones via a computer enabling the skin to be categorized by processing this combination of signals, notably by supervised learning from a reference base; and
Recommending one or more cosmetic products suited to the skin of the individual based on the combination of biomarkers revealed.

The recommendation is established on the basis of a decision tree that takes into account information relating to an irritated and/or infected skin condition, which translates into the following criteria, for example:
1—Information about the level of skin irritation or inflammation such as the presence of atopic dermatitis, eczema, rosacea, etc., by virtue of the following “skin” biomarkers:
   Biological markers: keratin (sample quality control), filaggrin, etc., IgE, CRP, IL-6, etc. ;
   CRP: may vary based on menstrual cycle, infection marker;
   If keratin marker is low=recent and overly aggressive scrubbing or exfoliation, peeling;

2—Information about the composition of the skin microbiota (level of skin colonization/contamination).

The microbiota is considered “normal” if colonization is of the “commensal” type, the microbiota being made up of expected microorganisms.

Contamination or infection will be detected if the biological marker of the target microorganism is elevated, for example:
Presence of acne if C. acnes is elevated;
Presence of infected atopic dermatitis (eczema) if the S. aureus marker is elevated;
Presence of rosacea if the S. epidermidis or Demodex marker is elevated; and
Risk of dandruff on the scalp if the Malassezia marker is elevated.

As far as skin microbiota is concerned, the analysis provides information on the balance between different populations of microorganism and can detect the existence of dysbiosis.

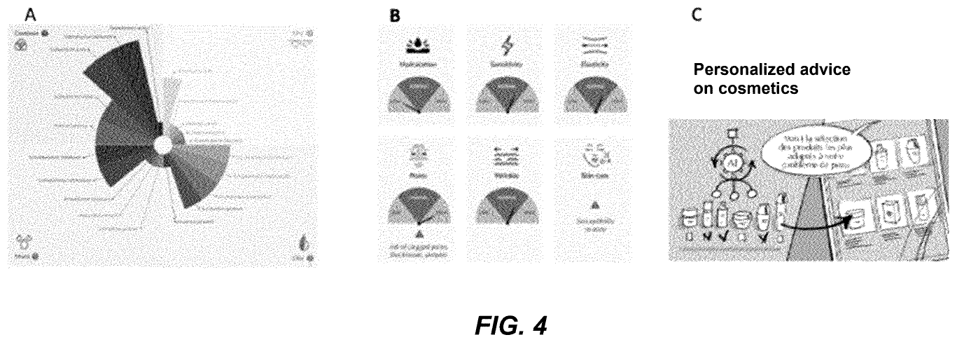

FIG. 4 shows the steps involved in this recommendation method.

### Non-Limiting Example of an Embodiment of the Present Disclosure

The present disclosure will be better understood on reading the following description, which concerns a non-limiting example embodiment, where:

A person notices the appearance of pimples on her face, she considers her skin to be rather oily, she has a skincare routine that suits her (advice from her mother, friends, beauticians etc.), she has heard about intestinal microbiota and its impact on health (stimulation of the immune system, protection against pathogenic germs that can irritate or even attack the digestive tract etc.), there are tests but this is not practical as you have to order a sampling kit from a website, which is fairly expensive (€250 to €500), take the sample at home and then send it to a laboratory for a “metagenomic” analysis, and then wait several weeks for a result and advice. You need to be highly motivated and patient.

With the device according to the present disclosure, all you have to do is visit a cosmetics store or a pharmacy (OTC cosmetics and personal care), or order it via a smartphone application. Although it is recommended to perform the test in the morning after having removed make-up and rinsed the skin of the face the day before, the test can also be performed extemporaneously (with the risk of identifying only the 3 or 4 main microorganisms in the microbiota, but this may be sufficient to confirm a suspicion of acne, for example). The device comes in the form of a kit with a simple skin sampling device: a small, spongy, absorbent, slightly abrasive device soaked in a solution to optimize sampling is applied, then this device is inserted into a tube containing a so-called “resuspension” (or “solubilization”) solution.

The solution flows into the ICFL device, which consists of a sheet of absorbent nitrocellulose (NC) to create a migration flow, with an entry zone above which is another sheet of NC, where a “detection conjugate” has been deposited. This detection conjugate is generally a monoclonal antibody directed against a target antigen, the analyte investigated, that is, a protein characteristic of the condition of the skin (structural protein—keratin, filaggrin, etc., —, inflammation—CRP, etc., —, the immune system—IL6, etc., —etc.) or of the microbiota (adhesion proteins fimbriae, curli, pili, etc., characteristic of the presence of the microbiota as a biofilm naturally formed on the skin, expressed by the 3 major bacteria Staphylococcus epidermidis, Cutibacterium acnes, Staphylococcus aureus, and other more minor bacteria—some common to all skin types and others specific to dry, or oily or moist skin). The detection conjugate is so called because a compound (gold microparticle, latex microbead, etc.) is bound to it, which enables the antigen-antibody interaction to be visualized.

After this initial interaction at the entrance to the deposition well, the flow progresses toward a detection zone on which a monoclonal capture antibody has been deposited, also directed against antigens characteristic of the condition of the skin and of the microbiota. Each capture antibody is deposited on a test line, the result of which indicates the presence or absence of the analyte.

In addition, there is always a control line, the result of which is used to validate correct migration and therefore validate the test. For there to be a reaction in this zone, the antibody bound to the membrane on the control line is directed against the conjugate.

Each ICFL device is intended for multiple analyte detection (requiring multiple “detection conjugate and capture antibody” pairs). This makes it possible to determine a skin condition profile (healthy, normal, dry, oily, etc.) associated with a microbiota profile (healthy, characteristic of dry skin, oily skin, susceptible to acne, dermatitis, etc.).

Reading and analysis are performed by smartphone image capture and interpreted by AI. The information provided by the biomarkers can also be combined with information provided by the individual.

### Example of Implementation of the Device for a Cosmetics Recommendation

The present disclosure thus enables different types of tests:
i. One-off test in 15 to 30 minutes (possible in store or at home in a “Home test”), either as a spontaneous process, or in reaction to an event: use of an irritating cosmetic or personal care product, appearance of solar erythema following prolonged exposure to the sun, or following a session in an artificial UV booth, or after a scrub or an exfoliation or a peeling that is too intense, or else the diagnosis of a skin pathology (the most common skin conditions being acne, eczema, psoriasis, scalp diseases (excluding alopecia), mycosis and nail diseases);
ii. Repeated, successive tests allow us to monitor the skin's general condition over time by associating it with the resident microbiota (skin-microbiota complex), which can lead to better management of exposure to the sun or artificial UV rays, or to spacing out or attenuating exfoliation or peeling practices;
iii. Depending on the profile obtained, the proposal of suitable products linking the skin's condition to an ad hoc treatment (dry skin=moisturizing routine, oily skin=cleansing exfoliation routine, thin and wrinkled skin=protective and nourishing cream routine, etc.) with associated “microbiota” information: either “probiotic” supplements to stimulate the skin and/or microbiota, or “compatible” products to avoid treating the skin alone to the detriment of the microbiota (a test campaign of the products versus microbiota microorganisms can enable a compatibility score to be calculated), or a skincare product that can improve an unpleasant syndrome (sensation of dry, oily, irritated skin, redness, etc.);
iv. To check the compatibility of products used routinely. To change the routine based on the evolution of the skin-microbiota complex with age (when the skin becomes thinner), climate (hot, humid summer versus cold, dry winter), pollution, etc.).

1. A personalized cosmetic recommendation method based on immunochromatographically characterizing, simultaneously and immediately after sampling, different biomarkers of the skin-microbiota complex of an individual comprising the following steps:
providing a sample of biological material taken from the surface of the skin and containing the constituent elements of the skin-microbiota complex, the skin-microbiota complex consisting of skin cells and microorganisms and other constituent elements of the skin microbiota;
suspending the skin-microbiota complex in a solubilization solution; and
pouring the solubilization solution into the transfer well of an immunochromatographic revealing device, the immunochromatographic revealing device comprising a support on which is arranged a transfer well configured to receive the solubilization solution, the well leading to a plurality of N revealing zones each having (i) an immunological detection reagent and (ii) an immunological capture reagent, and, the immunological revealing device comprising:
at least one immunological detection reagent specific for a skin cell biomarker and at least one immunological detection reagent specific to a biomarker of the skin microbiota;
the immunological detection reagents each comprise an antibody specific to a biomarker of skin cells or of the skin microbiota, conjugated to a colorimetric identification system;
the immunological capture reagents each comprise an antibody, bound to the support in each of the N revealing zones, capable of recognizing the biomarker present in the zone; and
at least one fool-proofing means for orienting the reading of the device;
incubating between 1 and 10 min in order to allow the cells of the skin-microbiota complex to react with the reagents on each of the N zones of the immunological revealing device;
processing the image consisting of the set of signals revealed on each of the N revealing zones via a computer enabling the skin-microbiota complex to be categorized by processing this combination of signals; and
recommending one or more cosmetic products based on the combination of biomarkers revealed;
wherein:
the number N of revealing zones is greater than or equal to 5 and comprises a) at least three zones for revealing three biomarkers of the skin microbiota, the biomarkers being bacteria of the genera Staphylococcus, Cutibacterium and Corynebacterium and b) at least one zone for revealing a skin cell biomarker selected from (i) a structural protein, (ii) an inflammatory biomarker or (iii) an allergy biomarker; and
the recommended cosmetic products are suited on the one hand to the skin type of the individual and are, on the other hand, compatible with the microbiota thereof.

2. The method of claim 1, wherein the recommendation of one or more cosmetic products is established on the basis of a decision tree based on the following criteria:
the physiological state of the skin, established by virtue of analyzing signals relating to skin cell biomarkers; and
the composition of the microbiota established by virtue of analyzing signals relating to biomarkers of the microbiota, including the balance between different populations of microorganisms.

3. The method of claim 1, wherein the biomarker analysis is a semi-quantitative analysis.

4. The method of claim 1, wherein the bacterium of the genus Staphylococcus is selected from Staphylococcus epidermidis and Staphylococcus aureus, the bacterium of the genus Cutibacterium is selected from Cutibacterium acnes and Cutibacterium granulosum and the bacterium of the genus Corynebacterium is selected from Corynebacterium xerosis and Corynebacterium kroppenstedtii.

5. The method of claim 1, wherein at least one of the skin microbiota-specific biomarkers corresponds to an epitope associated with biofilm-like behavior of the microbiota.

6. The method of claim 1, wherein at least one specific revealing zone of an additional biomarker of the skin microbiota is a microorganism selected from bacteria, yeasts, fungi and mites.

7. The method of claim 1, wherein the additional biomarker of the skin microbiota is selected from the following species:
(i) a bacterium selected from Staphylococcus epidermidis, Staphylococcus aureus, Staphylococcus hominis, Staphylococcus lugdunensis, Staphylococcus capitis, Cutibacterium acnes, Propionibacterium granulosum, Propionibacterium avidum, Corynebacterium tuberculostearicum, Corynebacterium simulans, Corynebacterium fastidiosum, Corynebacterium afermentans, Corynebacterium xerosis, Corynebacterium aurimucosum), Corynebacterium kroppenstedtii, Corynebacterium amycolatum, Streptococcus mitis, Streptococcus orali), Streptococcus pseudopneumoniae, Streptococcus sanguinis, Veillonella parvula, Micrococcus luteus, Enhydrobacter aerosaccus, Epidermophyton floccosum, Nannizzia nana, Nephroselmis olivacea, Cyanophora paradoxa, Aureoumbra lagunensis, Pycnococcus provasolii, Pyramimonas parkeae, Parachlorella kessleri, Aspergillus tubingensis, Zymoseptoria tritici, Tilletia Dermacoccus, Actinomyces;
(ii) a yeast selected from Malassezia globosa, Malassezia restricta, Malassezia furfur or Candida parapsilosisparapsilosis;
(iii) a fungus selected from Aspergillus tubingensis, Zymoseptoria tritici, Tilletia walkeri, Epidermophyton floccosum, Nannizzia nana, Nephroselmis olivacea, Cyanophora paradoxa, Aureoumbra lagunensis, Pycnococcus provasolii; and
(iv) a mite selected from Demodex folliculorum, Demodex brevis.

8. The method of claim 1, wherein the biomarker of the microbiota is a protein exposed on the surface of the microorganism selected from Panton-Valentine Leukocidin, porphyrins, s alginates, β-1,6-linked N-acetylglucosamine (PNAG), Bap family adhesins, type 4 pili, Self-associating autotransporters, intimins/invasin, CAMP factors, sialidases, dermatan-sulphate adhesins, endoglycoceramidases, GroEL chaperonins, SH3 domain-containing lipoprotein, Flp pili/fimbriae protein, DsA1 protein; Clf-Sdr family proteins, such as Bbp, FnBPs and CNA, SesJ protein, aureusimine, Bap family adhesins displaying an LPXTG C-terminal domain, IMPs, type 3 and 4 pili.

9. The method of claim 1, wherein the biomarkers of skin cells are selected from (i) a structural protein selected from keratin, filaggrin, loricrin, (ii) an inflammatory biomarker selected from C-Reactive Protein, interleukins IL-1beta, IL-4, IL-6, IL-8, IL-11, IL-12, tumor necrosis factor, interferon-gamma, granulocyte-macrophage colony-stimulating factor, transforming growth factor-beta and (iii) an allergy biomarker selected from immunoglobulins IgE, IgA and IgG.

10. The method of claim 1, wherein the skin cell biomarker is interleukin 1-beta.

11. The method of claim 1, wherein it comprises zones for specifically revealing the presence of bacteria of the genera Staphylococcus, Cutibacterium and Corynebacterium and of IL-1beta.

12. The method of claim 1, wherein the device is a lateral flow immunochromatographic device.

13. The method of claim 1, wherein the bacterial concentration of the solubilization solution after contact is made with the sampled biological material is of the order of 10⁴ CFU/mL.

14. (canceled)
